(19) United States
(12) Patent Application Publication
(10) Pub. No.: US 2016/0331733 A1
(43) Pub. Date: Nov. 17, 2016

(54) NEW USE OF ACLIDINIUM

(71) Applicant: Almirall, S.A., Barcelona (ES)

(72) Inventors: Diana JARRETA FERNANDEZ, Sant Feliu de Llobregat (Barcelona) (ES); Maria Esther GARCIA GIL, Sant Feliu de Llobregat (Barcelona) (ES)

(21) Appl. No.: 15/218,391

(22) Filed: Jul. 25, 2016

Related U.S. Application Data
(63) Continuation of application No. 14/652,817, filed on Jun. 17, 2015, now abandoned.
(60) Provisional application No. 61/750,952, filed on Jan. 10, 2013.

(30) Foreign Application Priority Data
Dec. 17, 2012 (EP) 12382507.7

(51) Int. Cl. A61K 31/439; A61K 45/06; A61K 9/00

(52) U.S. Cl. CPC A61K 31/439; A61K 9/0075; A61K 45/06

(57) ABSTRACT

The present disclosure provides aclidinium or any of its stereoisomers or mixture of stereoisomers, or a pharmaceutically acceptable salt or solvate thereof, for improving the quality of sleep in respiratory patients.

### FIELD OF THE INVENTION

The invention relates to a novel use of aclidinium, which can be advantageously used to improve physical activity in respiratory patients.

### BACKGROUND OF THE INVENTION

Respiratory diseases, such as asthma and chronic obstructive pulmonary disease (COPD), are a significant global health problem, with an increasing incidence throughout the world. They are usually characterised by an inflammatory dysfunction of the airways which results in bronchoconstriction.

In asthma inflammation is driven by exposure to a variety of triggers, including allergens and viruses, which activate components of both the innate and acquired immune responses. In COPD inflammation occurs primarily because of exposure to noxious particles and gases, in particular to cigarette smoke. Rather than a single pathologic condition, COPD is a term encompassing several disorders, such as chronic bronchitis or emphysema.

According to a scientific statement from the American Heart Association (Thompson, P D et al, Arterioscler Thromb Vasc Biol. 2003; 23: e42-e49), physical activity is defined as “any bodily movement produced by skeletal muscles that results in energy expenditure beyond resting expenditure”. This definition of physical activity includes activities such as activities of daily living, sports, and activities for personal fulfilment (Gimeno-Santos et al, Health and Quality of Life Outcomes, 2011; 9: 86).

In certain groups of respiratory patients, for example those with a more severe degree of the disease, physical activity is significantly reduced. This reduction of physical activity may contribute to a further deterioration of the health status of the patients and result in an exacerbation of the respiratory symptoms and/or the appearance of co-morbidities, such as cardiovascular or metabolic diseases in addition to deconditioning and muscle weakness.

Physical activity can be measured in terms of walking and/or running distance (steps or meters), walking and/or running time (min), standing time (min), sitting time (min), lying time (min), movement intensity during walking and/or running (meters/s2), calories expended or metabolic equivalents, skin temperature, heat flux (amount of heat dissipating from the body), galvanic skin response (electrical conductivity of the skin, which changes in response to sweat and emotional stimuli), among others.

Physical activity monitors are frequently used to estimate levels of daily physical activity and to investigate possible relationships between physical activity levels and clinical outcome. These devices use piezoelectric accelerometers, which measure body's acceleration, in one, two or three axes. The signal can be transformed into an estimate of energy expenditure using one of variety of algorithms, or summarized as activity counts or vector magnitude units (reflecting acceleration). With the information obtained in the vertical plane or through pattern recognition, steps and walking time can also be derived.

An example of a commonly used accelerometer is the multisensory armband device SenseWear™ Pro Armband (BodyMedia, Inc., Pittsburg, Pa., USA) (Watz H et al, Eur Respir J, 2009; 33: 262-272; Troosters T et al, The Open Respiratory Medicine Journal, 2011, 5, 1-9; O'Donnell D E et al, Respiratory Medicine, 2011, 105, 1030-1036; Waschki B et al, Respiratory Medicine, 2012, 106, 522-530).

Parameters of physical activity over time as determined by an accelerometer, include: 1) average number of steps per day; 2) average time (minutes) spent per day in at least moderate activity (defined as any physical activity>3 metabolic equivalents); 3) average active energy expenditure (Kcal spend in at least moderate activities); or 4) physical activity level (PAL).

Tiotropium is the first long-acting anticholinergic bronchodilator indicated as a maintenance treatment to relieve symptoms of COPD patients. Although tiotropium has been shown to produce sustained improvement in pulmonary function in patients with moderate to severe COPD, tiotropium has not statistically demonstrated improvements in physical activity of patients with COPD versus placebo (Sciurba F C et al, Am J Respir Crit Care Med 183, 2011, A1589).

It has now surprisingly been found that aclidinium significantly increases physical activity in daily life of respiratory patients, increasing thus overall quality of life.

Aclidinium has the chemical name 3(R)-(2-hydroxy-2,2-dithien-2-ylacetoxy)-1-(3-phenoxypropyl)-1-azoniabicyclo[2.2.2]octane and was first disclosed in WO 01/04118. It is a long-acting muscarinic receptor antagonist recently approved by the US Food and Drug Administration (FDA) and the European Medicines Agency (EMA) for the maintenance treatment to relieve respiratory symptoms in patients with COPD. Pharmaceutical compositions comprising aclidinium are described in EP2100598A1 and in EP2100599A1. However, until now, there is no disclosure about that aclidinium significantly increases physical activity in daily life of respiratory patients. Its effect in improving physical activity is an unexpected finding of this invention having regard to the lack of significant activity of tiotropium, the reference long-acting antimuscarinic drug currently in the market.

### SUMMARY OF THE INVENTION

The present invention provides aclidinium, or any of its steroisomers or mixture of stereoisomers, or a pharmaceutically acceptable salt or solvate thereof, for use in improving the physical activity in respiratory patients, in particular daily physical activity.

Preferably, aclidinium is in the form of a salt with an anion X⁻. Most preferably, the anion X⁻ is bromide.

In a preferred embodiment, the respiratory patient suffers from a disease selected from acute or chronic bronchitis, emphysema, asthma and chronic obstructive pulmonary disease, preferably asthma and chronic obstructive pulmonary disease, most preferably chronic obstructive pulmonary disease.

In another embodiment, aclidinium is administered as a pharmaceutical composition suitable for inhalation, preferably in the form of a dry powder. The composition can be administered by means of any inhaler device, more preferably the Genuair® device.

Typically, a dry powder formulation comprises a pharmaceutically acceptable carrier selected from mono-, di- or polysaccharides and sugar alcohols. Preferably, the carrier is lactose, more preferably lactose monohydrate, even more preferred alpha-lactose monohydrate.

Aclidinium is administered at least once a day, preferably in the morning or in the evening. More preferably aclidinium is administered twice daily, i.e. two oral inhalations per day. In a most preferred embodiment aclidinium is administered twice daily, one in the morning and another one in the evening. In a preferred embodiment, the aclidinium is administered at least during 3 weeks, preferably at least during more than 52 weeks.

The effective dose of aclidinium to be used per inhalation is the equivalent to a metered nominal dose from 100 to 1000 micrograms of aclidinium bromide per inhalation in a dry powder for inhalation, more preferably 200 or 400 micrograms of aclidinium bromide per inhalation. In a most preferred embodiment the effective dose of aclidinium is the equivalent to a metered nominal dose of 400 micrograms of aclidinium bromide per inhalation.

In a particular embodiment, the effective dose of aclidinium to be used per inhalation is the equivalent to a metered nominal dose of 400 micrograms of aclidinium bromide per inhalation and/or a metered nominal dose of 343 micrograms of aclidinium per inhalation.

In another particular embodiment, the effective dose of aclidinium to be used per inhalation is the equivalent to a delivered dose (the dose leaving the mouthpiece of the inhaler device) of 375 micrograms of aclidinium bromide per inhalation and/or a delivered dose of 322 micrograms of aclidinium per inhalation. The delivered dose can be measured using standard techniques known to those skilled in the art.

In another preferred embodiment, aclidinium is co-administered with an additional medication suitable for the treatment of respiratory diseases, selected for example from one or more of the following: corticosteroids, beta-adrenergic agonists, PDE4 inhibitors, antihistamines, anti-IgE antibodies, leukotriene D4 inhibitors, inhibitors of egfr-kinase, p38 kinase inhibitors and/or NK1-receptor antagonists. The additional medications can be present in the same pharmaceutical composition as aclidinium or in separate pharmaceutical compositions. Preferably, the additional medication is selected from corticosteroids, beta-adrenergic agonists and/or PDE4 inhibitors.

The improvement by aclidinium of the physical activity in respiratory patients can be measured by observing the improvement of one or more of the following:

a) average number of steps per day;

b) minutes of moderate activity per day;

c) average active energy expenditure (expressed in Kcal); or

d) physical activity level (PAL).

The physical activity level (PAL) can be obtained by dividing the total daily energy expenditure by whole-night sleeping energy expenditure. A physical activity level ≧1.70 defines an active person, 1.40-1.69 defines a predominantly sedentary person, and <1.40 defines a very inactive person. A person with a physical activity level of 1.2 is usually chair-or bed-bound.

The invention further provides a pharmaceutical composition comprising aclidinium for improving the physical activity in respiratory patients.

The invention further provides the use of aclidinium in the manufacture of a medicament for improving the physical activity in respiratory patients.

The invention further provides a method of improving the physical activity in respiratory patients, which method comprises administering to said patient an effective amount of aclidinium, as defined above.

a) reduced average number of steps per day;

b) reduced minutes of moderate activity per day;

c) reduced average active energy expenditure; or

d) reduced physical activity level (PAL).

The invention further provides aclidinium, as defined above, in the manufacture of a medicament for treating a patient suffering from a respiratory disorder, preferably asthma or chronic obstructive pulmonary disease, wherein the patent presents a reduced physical activity. Preferably, the reduced physical activity involves one or more of the following:

a) reduced average number of steps per day;

b) reduced minutes of moderate activity per day;

c) reduced average active energy expenditure; or

d) reduced physical activity level (PAL).

### DETAILED DESCRIPTION OF THE INVENTION

Typically, aclidinium is administered in the form of a salt with an anion X⁻, wherein X⁻ is a pharmaceutically acceptable anion of a mono or polyvalent acid. More typically, X⁻ is an anion derived from an inorganic acid, such as hydrochloric acid, hydrobromic acid, sulphuric acid and phosphoric acid, or an organic acid such as methanesulphonic acid, acetic acid, fumaric acid, succinic acid, lactic acid, citric acid or maleic acid. Most preferably aclidinium is in the form of aclidinium bromide.

Aclidinium bromide is a white powder with a molecular formula of C₂₆H₃₀NO₄S₂Br and a molecular mass of 564.56. It is very slightly soluble in water and ethanol and sparingly soluble in methanol.

The compound of the invention may exist in both unsolvated and solvated forms. The term solvate is used herein to describe a molecular complex comprising a compound of the invention and an amount of one or more pharmaceutically acceptable solvent molecules. The term hydrate is employed when said solvent is water. Examples of solvate forms include, but are not limited to, compounds of the invention in association with water, acetone, dichloromethane, 2-propanol, ethanol, methanol, dimethylsulfoxide (DMSO), ethyl acetate, acetic acid, ethanolamine, or mixtures thereof. It is specifically contemplated that in the present invention one solvent molecule can be associated with one molecule of the compounds of the present invention, such as a hydrate.

The words “treatment” and “treating” are to be understood as embracing amelioration of symptoms of a disease or condition and/or elimination or reduction of the cause of the disease or condition and/or prevention of the appearance of the disease or its symptoms.

The term “therapeutically effective amount” refers to an amount sufficient to effect treatment when administered to a patient in need of treatment.

Aclidinium can also be used in combination with other drugs known to be effective in the treatment of the diseases or the disorders indicated above. For example aclidinium can be combined with corticosteroids or glucocorticoids, beta-adrenergic agonists, PDE4 inhibitors, antihistamines, anti-IGE antibodies, leukotriene D4 antagonists, inhibitors of egfr kinase, p38 kinase inhibitors and/or NK-1 receptor agonists.

Corticosteroids that can be combined with aclidinium in the present invention particularly include those suitable for administration by inhalation in the treatment of respiratory diseases or conditions, e.g., prednisolone, methylprednisolone, dexamethasone, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone furoate, rimexolone, prednisolone farnesylate, ciclesonide, deprodone propionate, fluticasone propionate, fluticasone furoate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate and hydrocortisone probutate. Budesonide, fluticasone propionate and mometasone furoate are especially preferred.

Beta-adrenergic agonists that can be combined with aclidinium in the present invention particularly include β2 adrenergic agonists useful for treatment of respiratory diseases or conditions, for example, selected from the group consisting of arformoterol, bambuterol, bitolterol, broxaterol, carbuterol, clenbuterol, dopexamine, fenoterol, formoterol, hexoprenaline, ibuterol, isoprenaline, mabuterol, meluadrine, nolomirole, orciprenaline, pirbuterol, procaterol, reproterol, ritodrine, rimoterol, salbutamol, salmeterol, sibenadet, sulfonterol, terbutaline, tulobuterol, vilanterol, olodaterol, KUL-1248, abediterol, carmoterol and indacaterol, in free or pharmaceutically acceptable salt form. Preferably, the β2 adrenergic agonist is a long-acting β2 adrenergic agonist, e.g., selected from the group consisting of formoterol, salmeterol, carmoterol, vilanterol, olodaterol, abediterol and indacaterol in free or pharmaceutically acceptable salt form.

PDE4 inhibitors that can be combined with aclidinium in the present invention include denbufylline, rolipram, cipamfylline, arofylline, filaminast, piclamilast, mesopram, drotaverine hydrochloride, lirimilast, roflumilast, cilomilast, 6-[2-(3,4-Diethoxyphenyl)thiazol-4-yl]pyridine-2-carboxylic acid, (R)-(+)-4-[2-(3-Cyclopentyloxy-4-methoxyphenyl)-2-phenylethyl]pyridine, N-(3,5-Dichloro-4-pyridinyl)-2-[1-(4-fluorobenzyl)-5-hydroxy-1H-indol-3-yl]-2-oxoacetamide, 9-(2-Fluorobenzyl)-N6-methyl-2-(trifluoromethyl)adenine, N-(3,5-Dichloro-4-pyridinyl)-8-methoxyquinoline-5-carboxamide, N-[9-Methyl-4-oxo-1-phenyl-3,4,6,7-tetrahydropyrrolo[3,2,1-jk][1,4]benzodiazepin-3(R)-yl]pyridine-4-carboxamide, 3-[3-(Cyclopentyloxy)-4-methoxybenzyl]-6-(ethylamino)-8-isopropyl-3H-purine hydrochloride, 4-[6,7-Diethoxy-2,3-bis(hydroxymethyl)naphthalen-1-yl]-1-(2-methoxyethyl)pyridin-2(1H)-one, 2-carbomethoxy-4-cyano-4-(3-cyclopropylmethoxy-4-difluroromethoxyphenyl)cyclohexan1-one, cis [4-cyano-4-(3-cyclopropylmethoxy-4-difluoromethoxyphenyl)cyclohexan-1-ol, ONO-6126 (Eur Respir J 2003, 22(Suppl. 45): Abst 2557) and the compounds claimed in the PCT patent application numbers WO 03/097613, WO 2004/058729, WO 2005/049581, WO 2005/123692, WO 2005/123693 and WO 2010/069504.

Aclidinium for use in the present invention may be administered by any suitable route to provide local antimuscarinic action. It is preferably administered by inhalation, e.g., as a powder, spray, or aerosol, preferably as a dry powder. Pharmaceutical compositions comprising aclidinium may be prepared using conventional diluents or excipients and techniques known in the galenic art.

Medicaments for administration in a dry powder for inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means, e.g. by micronisation or supercritical fluid techniques. The desired fraction may be separated out by air classification or sieving. Preferably, the particles will be crystalline.

Achieving high dose reproducibility with micronised powders is difficult because of their poor flowability and extreme agglomeration tendency. To improve the efficiency of dry powder compositions, the particles should be large while in the inhaler, but small when discharged into the respiratory tract. Thus, an excipient, for example a mono-, di- or polysaccharide or sugar alcohol, such as lactose, mannitol or glucose is generally employed. The particle size of the excipient will usually be much greater than the inhaled medicament within the present invention. When the excipient is lactose it will typically be present as lactose particles, preferably crystalline alpha lactose monohydrate, e.g., having an average particle size range of 20-1000 μm, preferably in the range of 90-150 μm. In one embodiment, the lactose particles for use in formulations of the invention have a d10 in the range of 90-160 μm, a d50 in the range of 170-270 μm, and d90 in the range of 290-400 μm.

Suitable lactose materials for use in the present invention are commercially available, e.g., from DMW Internacional (Respitose GR-001, Respitose SV-001, Respitose SV-003); Meggle (Capsulac 60, Inhalac 70, Capsulac 60 INH); and Borculo Domo (Lactohale 100-200, Lactohale 200-300, and Lactohale 100-300).

The ratio between the lactose particles and aclidinium by weight will depend on the inhaler device used, but is typically, e.g., 5:1 to 200:1, preferably 25:1 to 150:1, more preferably 30:1 to 70:1.

In a preferred embodiment, the aclidinium is administered in the form of a dry powder formulation of aclidinium bromide in admixture with lactose, in a ratio by weight of aclidinium to lactose of 1:50 to 1:150, suitable for administration via a dry powder inhaler, wherein the aclidinium particles have an average particle size of from 2 to 5 μm in diameter, e.g., less than 3 μm in diameter, and the lactose particles have have a d10 of 90-160 μm, a d50 of 170-270 μm, and d90 of 290-400 μm.

Dry powder compositions for topical delivery to the lung by inhalation may, for example, be presented in capsules and cartridges of for example gelatine or blisters of for example laminated aluminium foil, for use in an inhaler or insufflator. Each capsule or cartridge may generally contain between 0.001-200 mg, more preferably 0.01-100 mg of active ingredient or the equivalent amount of a pharmaceutically acceptable salt thereof. Alternatively, the active ingredient (s) may be presented without excipients.

Packaging of the formulation may be suitable for unit dose or multi-dose delivery. In the case of multi-dose delivery, the formulation can be pre-metered or metered in use. Dry powder inhalers are thus classified into three groups: (a) single dose, (b) multiple unit dose and (c) multi dose devices.

Aclidinium is preferably administered with a multi-dose inhaler, more preferably with the Genuair® device (formerly known as Novolizer SD2FL), which is described the PCT patent application numbers WO 97/000703, WO 03/000325 and WO 2006/008027 and in Chrystyn H et al, Int J Clin Pract, March 2012, 66, 3, 309-317 (first published online on 16 Feb. 2012).

### EXAMPLE 1

In a Phase III randomized, double-blind, placebo controlled, 2 period crossover trial, patients with moderate to severe COPD received a dose of aclidinium equivalent to a metered nominal dose of 400 micrograms of aclidinium bromide per inhalation twice-daily (in the morning, 9 am, and in the evening, 9 pm) and placebo for two periods of 3 weeks, with a washout period of 2 weeks between treatment periods.

Patients were randomised to receive either a dose of aclidinium equivalent to a metered nominal dose of 400 micrograms of aclidinium bromide per inhalation twice-daily in the first period followed by placebo in the second period, or to receive placebo in the first period followed by aclidinium bromide 400 micrograms twice-daily in the second period. Both aclidinium bromide and placebo were administered with a Genuair® multidose dry powder inhaler.

Physical activity (as minutes of at least moderate activity per day and as average active energy expenditure expressed in Kcal) was measured in the last week of the treatment with aclidinium and placebo using a multisensor armband (SenseWear™ Pro Armband; BodyMedia, Pittsburg, Pa., USA), was worn on the upper right over the triceps muscle, according to the method described in Watz Bet al, Eur Respir J, 2009; 33: 262-272. The multisensory armband incorporates a biaxial accelerometer that records steps per day and physiological sensors of energy expenditure.

A valid period of measurement was defined as 5 days of measurement with the patients wearing the accelerometer at least 22 hours per day.

The Intention-to-Treat (ITT) population included 109 patients.

Results are displayed in Table 1.

TABLE 1
Change from baseline in physical activity parameters

| | Change from baseline at week 3 | | Δ Compar- |
|---|---|---|---|
| | Placebo | Aclidinium | ison versus |
| Parameter | (n = 83) | (n = 85) | placebo |
| Duration of at least | −5.9 (SE = 4.1) | 4.2 (SE = 4.1) | 10 (p < 0.05) |
| moderate activity | | | |
| (minutes) | | | |
| Daily active energy | −32.7 (SE = 21.0) | 21.9 (SE = 20.8) | 55 (p < 0.05) |
| expenditure (Kcal) | | | |

n = number of patients in the analysis
SE = standard error

As it can be observed in Table 1, after 3 weeks of treatment, 400 micrograms of aclidinium bromide per inhalation twice-daily showed statistically significant increases in the adjusted mean change from baseline compared to placebo in the duration of at least moderate activity (10 minutes; p<0.05 versus placebo). Moderate activity defined as any physical activity >3 metabolic equivalents.

Aclidinium bromide also showed statistically significant increases in the adjusted mean change from baseline compared to placebo in daily active energy expenditure activity (55 Kcal; p<0.05 versus placebo)

These phase III results demonstrate a remarkable improvement in physical activity produced by aclidinium, which was not observed with tiotropium, the reference standard in COPD treatment, in previous trials (Sciurba F C et al, Am J Respir Crit Care Med 183, 2011, A1589).

1. A method of improving the physical activity in a patient in need thereof, comprising:
administering to the patient a pharmaceutical composition comprising aclidinium, any of its stereoisomers, or mixture of stereoisomers, or a pharmaceutically acceptable salt or solvate thereof.

2. The method according to clai wherein the aclidinium is in the form of aclidinium bromide.

3. The method according to claim 1, wherein the patient suffers from asthma or chronic obstructive pulmonary disease.

4. The method according to claim 1, wherein the aclidinium is in the form of a dry powder formulation suitable for inhalation.

5. The method according to claim 4, wherein the formulation is administered in metered nominal dose of aclidinium equivalent to an amount of aclidinium bromide ranging from 100 micrograms to 1000 micrograms.

6. The method according to claim 4, wherein the formulation is administered in metered nominal dose of aclidinium equivalent to 400 micrograms of aclidinium bromide per inhalation and/or a metered nominal dose of 343 micrograms of aclidinium per inhalation.

7. The method according to claim 4, wherein the formulation is administered in delivered dose of aclidinium equivalent to 375 micrograms of aclidinium bromide per inhalation and/or a delivered dose of 322 micrograms of aclidinium per inhalation.

8. The method according to claim 1, wherein the composition comprising aclidinium is administered at least once per day.

9. The method according to claim 1, wherein the composition comprising aclidinium is administered with a therapeutically effective amount of at least one other medication chosen from corticosteroids, beta-adrenergic agonists, and PDE4 inhibitors.

10. The method according to claim 1, wherein the physical activity is improved by increasing at least one of the following:
a) average number of steps per day;
b) minutes of moderate activity per day;
c) average active energy expenditure; or
d) physical activity level.

11-13. (canceled)

14. The method according to claim 1, wherein the patient suffers from a respiratory disorder and wherein the patient presents a reduced physical activity.

15. The method according to claim 14, wherein the reduced physical activity involves at least one of the following:
a) reduced average number of steps per day;
b) reduced minutes of moderate activity per day;
c) reduced average active energy expenditure; or
d) reduced physical activity level (PAL).

16. (canceled)

17. (canceled)

18. The method according to claim 5, wherein the formulation is administered in a metered nominal dose of aclidinium equivalent to an amount of aclidinium bromide ranging from 200 micrograms to 400 micrograms per inhalation.

19. The method according to claim 5, wherein the metered nominal dose of aclidinium is equivalent to 200 micrograms of aclidinium bromide per inhalation.

20. The method according to claim 5, wherein the metered nominal dose of aclidinium is equivalent to 400 micrograms of aclidinium bromide per inhalation.

21. The method according to claim 8, wherein the composition prising aclidinium is administered twice daily.

22. The method according to claim 14, wherein the respiratory disorder is asthma or chronic obstructive pulmonary disease.
